(19) United States
(12) Patent Application Publication
(10) Pub. No.: US 2009/0082662 A1
(43) Pub. Date: Mar. 26, 2009

(54) MRI BREAST IMAGE MAGNET STRUCTURE

(76) Inventor: Henry M. Israel, Bnei Brak (IL)

Correspondence Address: David Klein;DEKEL PATENT LTD., Room 27, 18 Menuha VeNahala Street, REHOVOT, IL

(21) Appl. No.: 12/234,661

(22) Filed: Sep. 21, 2008

Related U.S. Application Data
(60) Provisional application No. 60/974,627, filed on Sep. 24, 2007.

(51) Int. Cl. A61B 5/055; G01R 33/483

(52) U.S. Cl. 600/421; 324/309

(57) ABSTRACT

Apparatus for producing magnetic resonance (MR) images of a subject over a limited volume, including a patient table, and a shielded unit housed under the patient table, the shielded unit including an RF pulse generator module, a magnet assembly for producing a static magnetic field in magnetic structure including a shaped magnetic “C” core, a gradient field generating assembly for generating magnetic field gradients in two orthogonal planes for producing MR images, a radiofrequency (RF) coil assembly for transmitting a broad band radiofrequency pulse and a RF field matching structure to simulate an infinite volume to the RF coil assembly, wherein the RF coil assembly detects a plurality of MR signals induced from a subject in response to RF pulses from the RF pulse generator.

### CROSS-REFERENCE TO RELATED APPLICATIONS

This application claims priority under 35 USC §119 to U.S. Provisional Patent Application 60/974,627, filed Sep. 24, 2007, which is incorporated herein by reference.

### FIELD OF THE INVENTION

The present invention relates generally to a method and system for medical imaging, and particularly to a method and system for restricted volume imaging using a Magnetic Resonance Imaging (MRI) system, employing a stationary patient table and an electro magnetic field restricted to the area to be imaged. The present invention relates to imaging of the breast, in particular, and may extended to image extremities such as arms and legs or other constricted volumes like the prostrate. The system is an open structure allowing easy access to the imaged area for a hand guided biopsy or treatment.

### BACKGROUND OF THE INVENTION

Nuclear magnetic resonance imaging (“MRI”) is utilized for scanning and imaging biological tissue as a diagnostic aid, and is one of the most versatile and fastest growing modalities in medical imaging. As part of the MRI process, the subject patient is placed in a magnetic field. An assembly that may be closed or open creates the magnetic field.

Closed magnet assemblies have an enclosed workspace in which there is a controlled magnetic field. Open magnet assemblies have two spaced-apart magnet poles separated by a gap, with a working magnetic field volume located within the gap. The magnetic field produced within the workspace is applied to the subject tissue. The resulting nuclear magnetic resonance (“NMR”) in the tissue is unique for any given element at a given magnetic field. The magnetic field is controlled over the working volume to provide a unique field at each location in three-space using a number of coils to straighten and control the raw field produced within the structure. The working volume is excited and the resonant frequency read by local detectors.

The NMR data is then plotted into the frequency domain, K-Space, and mathematically transformed into real space to produce an image of the tissue.

Conventionally, the elements of the imaging apparatus are sized and arranged to image an entire human body during a scan. Recently, scanning devices have been developed to facilitate imaging only a particular anatomical area of interest of the subject patient, rather than the patient's entire body. For example, such devices can be used to scan only an extremity or joint of the patient. The devices are designed such that the dimensions of the magnet gap accommodate the extremity, such as an arm or leg, or joint, such as an elbow, knee, wrist, or ankle.

Conventional extremity scanners, however, have a major drawback in that, due to design constraints, sufficient scanning field strength is not provided to adequately image the target body part. Typically, the usable field within the gap is provided at a strength of no greater than 0.2 Tesla, which may limit some imaging applications. At least one design provides a larger field strength, but the structural design is such that weight-bearing scans are not possible, so the applications for this design are limited. In fact, most conventional designs require that an extremity to be scanned must be placed inside a small cylinder or other enclosed space using a patient handling device. Most patients are uncomfortable and become fidgety when confined in this manner, making it hard to obtain meaningful diagnostic information. In addition, direct hand delivered treatment or biopsy under MRI control is impossible in the present system configurations.

There is therefore a need for a scanner with magnet structure design that has dimensions suitable for use in imaging an extremity or particular portion of a subject's body, while still providing field strength within the magnetic field volume that will allow for clear imaging of the subject tissue. The design should provide ample room for the extremity so that the patient is comfortable. The magnet structure should also be constructed such that it can enable weight-bearing scans and biopsy, providing even more diagnostic flexibility.

### SUMMARY OF THE INVENTION

The present invention seeks to provide imaging apparatus for producing magnetic resonance (MR) images of a subject over a limited volume, as described more in detail hereinbelow.

In accordance with non-limiting embodiments of the present invention, the apparatus includes a magnet assembly for producing a static magnetic field in a shaped steel “C” core, a gradient field generating assembly for generating magnetic field gradients in at least two orthogonal planes for producing MR images and a radiofrequency (RF) coil assembly for transmitting a broad band radiofrequency pulse and a RF field matching structure to simulate an infinite volume to the RF coils. The RF coils detect a plurality of magnetic resonance (MR) signals induced from the subject in response to the RF pulse and the matching structure reduces the effect of reflections from the magnetic structure. The apparatus further includes a device for supporting the subject with respect to the magnet assembly and a plurality of digital receivers for receiving the plurality of MR signals. The receivers are adapted to be adjusted in phase and frequency to produce parallel spectral levels as inputs to three-dimensional K-space. A digital processor is further incorporated to transform the K-space date to an image.

Further, the open magnetic structure and speed of processing and producing an image allow real time, manual control of treatment and biopsies. The patient support allows static breast imaging using a horizontal table with breast locating receptacles. Further variations in the support structure allow imaging of extremities such as arms and legs with detail, weight bearing, and studying the finger, wrist, elbow, knee and ankle joints.

A method and apparatus for producing an image from a defined volume of interest, typically the breast, using a Magnetic Resonance Imaging (MRI) system are provided. The apparatus includes a base magnet assembly, at least one assembly capable of generating at least two orthogonal gradient magnetic fields and at least one radiofrequency coil. The apparatus additionally includes at least one field smoothing structure and radiofrequency (RF) field matching structure. A plurality of MR signals are detected from the RF coils, as the MR fields are translated in three-space and may be sent to the plurality of synthesized digital receivers. Each of the receivers may be configured either in phase or frequency. A plurality of respective sub-images are computed corresponding to the plurality MR signals for each of the receivers and for the given field-of-view (FOV) as desired. Combining the respective sub-images may form a composite image of the volume of interest.

There is provided in accordance with an embodiment of the present invention apparatus for producing magnetic resonance (MR) images of a subject over a limited volume, including a patient table, and a shielded unit housed under the patient table, the shielded unit including an RF pulse generator module, a magnet assembly for producing a static magnetic field in magnetic structure including a shaped magnetic “C” core, a gradient field generating assembly for generating magnetic field gradients in at least two orthogonal planes for producing MR images, a radiofrequency (RF) coil assembly for transmitting a broad band radiofrequency pulse and a RF field matching structure to simulate an infinite volume to the RF coil assembly, wherein the RF coil assembly detects a plurality of MR signals induced from a subject in response to RF pulses from the RF pulse generator.

In accordance with an embodiment of the present invention the matching structure reduces the effect of reflections from the magnetic structure.

Further in accordance with an embodiment of the present invention the matching structure includes a first layer adjacent the magnet structure and made of an absorbent material and multilayers of material that have different dielectric constants so as to match free air impedance to impedance of the absorbent material, a second layer which is a vertical gradient field source, and a third layer which is a horizontal gradient field source.

In accordance with an embodiment of the present invention a plurality of digital receivers are provided for receiving the plurality of MR signals. The digital receivers may be adjusted in phase and frequency to produce parallel spectral levels as inputs to K-space. A digital processor may be provided that transforms the K-space date to an image.

### BRIEF DESCRIPTION OF THE DRAWINGS

The present invention will be understood and appreciated more fully from the following detailed description, taken in conjunction with the drawings in which:

FIG. 1 is a simplified illustration of apparatus for producing magnetic resonance (MR) images of a subject over a limited volume, constructed and operative in accordance with an embodiment of the present invention.

FIG. 2 is a simplified illustration of a RF field coil where the magnetic field is limited to imaged volume of the embodiment shown in FIG. 1, in accordance with an embodiment of the present invention.

FIG. 3 is a simplified illustration of a magnet assembly for limited image system of the embodiment shown in FIG. 1, in accordance with an embodiment of the present invention.

FIGS. 4A and 4B are simplified illustrations of flux density and flux lines, respectively, of a working magnetic field of the embodiment shown in FIG. 1, in accordance with an embodiment of the present invention.

FIG. 5 is a simplified illustration of a 2D field gradient array using orthogonal linier elements, in accordance with an embodiment of the present invention.

FIG. 6 is a simplified illustration of a 2D field gradient array using orthogonal current feed elements, in accordance with an embodiment of the present invention.

FIG. 7 is a representation of the RF field matching and termination element, in accordance with an embodiment of the present invention.

FIG. 8 is a detail of K-Space filling of the embodiment shown in FIG. 1.

FIG. 9 is a fast vertical breast image of the embodiment shown in FIG. 1.

FIG. 10 is a fast lateral projection with computer-generated detail of the embodiment shown in FIG. 1.

FIG. 11 is a fast lateral projection including biopsy needle and detail of the embodiment shown in FIG. 1.

### DETAILED DESCRIPTION OF EMBODIMENTS

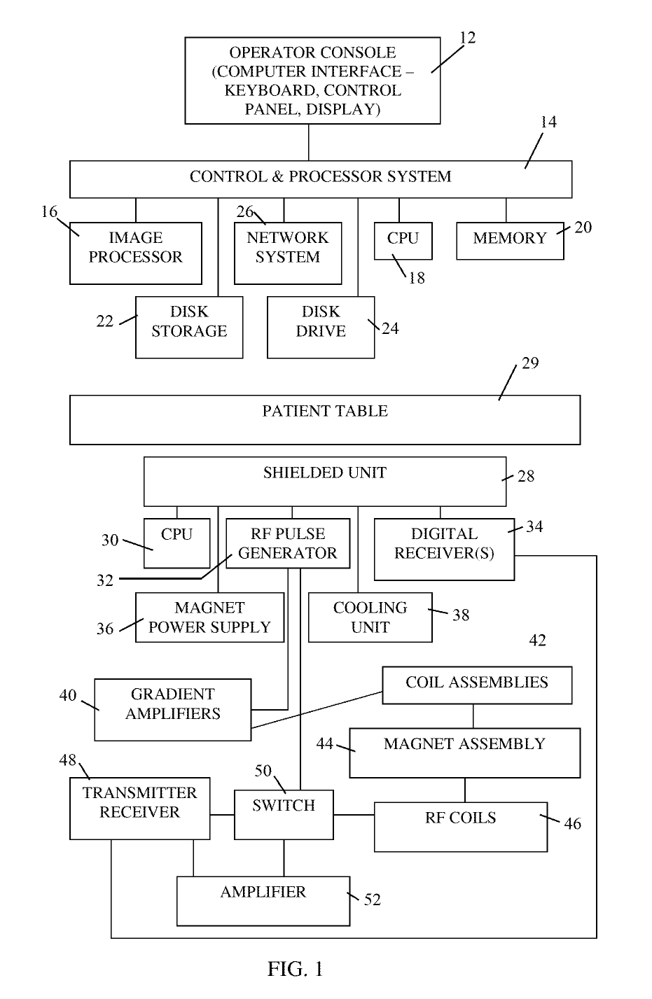

Reference is now made to FIG. 1, which illustrates apparatus for producing magnetic resonance (MR) images of a subject over a limited volume, constructed and operative in accordance with a non-limiting embodiment of the present invention.

FIG. 1 illustrates a simplified block diagram of a system for producing MR images in accordance with embodiments of the present invention. In the illustrated embodiment, the system is a MR imaging system designed for the stand-alone screening of breasts for anomalies. The MRI system could be, for example, adapted for performing breast biopsies. In another embodiment the MRI system can perform imaging and treatment of most other extremities.

The operation of the system is controlled from an operator console 12 consisting of a computer interface, including keyboard and control panel and a display. The console 12 communicates through links with a separate control and processor (also known as computer or control) system 14 that enables an operator to control the production and display images on the screen. The system 14 includes a number of modules, which communicate with each other. These include, without limitation, an image processor module 16, a CPU module 18, and a memory module 20, known in the art as a frame buffer for storing image data arrays. The computer system 14 is linked to disk storage 22 and a disk drive 24 for storage and mechanical transport of image data and programs, and it communicates with a network system 26 through a high-speed link.

The system as a stand-alone device is divided into two sections: one, the operator control and interface, and the second, a shielded unit 28 housed under a patient table 29. The shielded unit 28 includes a CPU (central processing unit) module 30, a RF pulse generator module 32, a broadband digital receiver 34 and the magnet power supply 36 and cooling unit 38. The control system receives commands from the operator, which indicate the scan sequence that is to be performed, and controlling the RF pulse generator output power, frequency, bandwidth and weighting, antenna configuration, receiver frequency, number of output channels and their bandwidths, and sets other system components to carry out the desired scan sequence. The controller produces a data report of the desired and accomplished sequence, indicating the timing, strength, and shape of the RF pulses produced, and the timing of and length of the data acquisition window. The controller module connects to a set of gradient amplifiers, if needed, to indicate the timing and shape of the gradient pulses to be produced during the scan. The controller module connects to all the needed safety devices on the system, reporting status and control parameters and providing safety alarms.

The gradient waveforms produced by the pulse generator module are applied to a gradient amplifier system 40 comprised of x and y amplifiers. Each gradient amplifier excites a corresponding coil in a coil assembly 42 generally designated to produce the magnetic field gradients used for position encoding acquired signals. The coil assembly 42 forms part of a magnet assembly which includes at least one polarizing magnet 44 and RF coils 46. It is noted that in this embodiment the RF and gradient coils are combined and include a field termination assembly. The working volume is that volume within the magnet assembly for receiving the part to be imaged and includes a patient volume. As used herein, the working volume of a MRI scanner is defined generally as a zone inside the patient volume where homogeneity of main, gradient and RF fields are within known, acceptable ranges for imaging.

A transceiver module 48 in the system produces pulses that are amplified by an RF amplifier and coupled to the RF coil by a transmit/receive (T/R) switch 50. In an embodiment of the present invention, the transceiver module 48 comprises a plurality of virtual digital receivers that will be discussed in greater detail. The resulting signals radiated by the excited nuclei within the subject may be sensed by the same RF coil 46 and coupled through the transmit/receive switch 50 to a preamplifier 52. The amplified MR signals are demodulated, filtered, and digitized in the receiver section of the transceiver 48. The transmit/receive switch 50 is controlled by a signal from the pulse generator module 32 to electrically connect the RF amplifier to the coil 46 during the transmit mode and to connect the preamplifier during the receive mode. The transmit/receive switch may also enable a separate RF coil (for example, a breast coil or surface coil) to be used as needed.

It is to be appreciated that RF coil 46 is configured to be operable for MRI scanning as described below, in which a subject is positioned along the z-axis. As used herein, “adapted to”, “configured” and the like refer to mechanical or structural connections between elements to allow the elements to cooperate to provide a described effect; these terms also refer to operation capabilities of electrical elements such as analog or digital computers or application specific devices (such as an application specific integrated circuit (ASIC)) that is programmed to perform a sequel to provide an output in response to given input signals. The MR signals picked up by the RF coil 46 are digitized by the transceiver module 48 and transferred to digital receiver module 34. The signals from the coils are phase shifted, weighted and summed to provide plane sensitive receiver inputs. The receiver inputs are processed to produce parallel output data streams for placement into the K-space memory. The preferred K-space entry method is, a pixel averaging, radial method.

When the scan is completed and an entire array of data has been acquired in the K-space memory module, an array processor operates to Fourier transform the data into an array of image data. These image data are conveyed to the computer system where they are displayed and stored in the disk memory. In response to commands received from the operator the images may be further processed by the image processor and conveyed to the operator and presented on the display. As will be discussed with reference to embodiments below, the image processor may perform further processing of the acquired MR image data as needed.

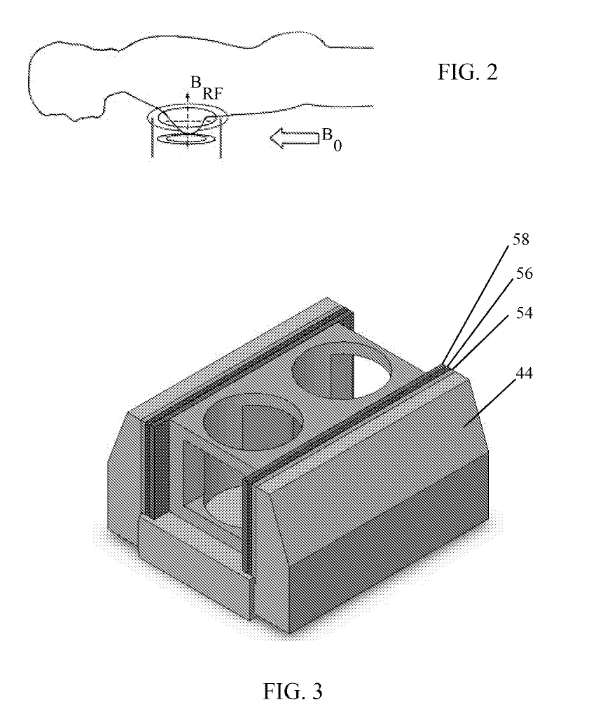

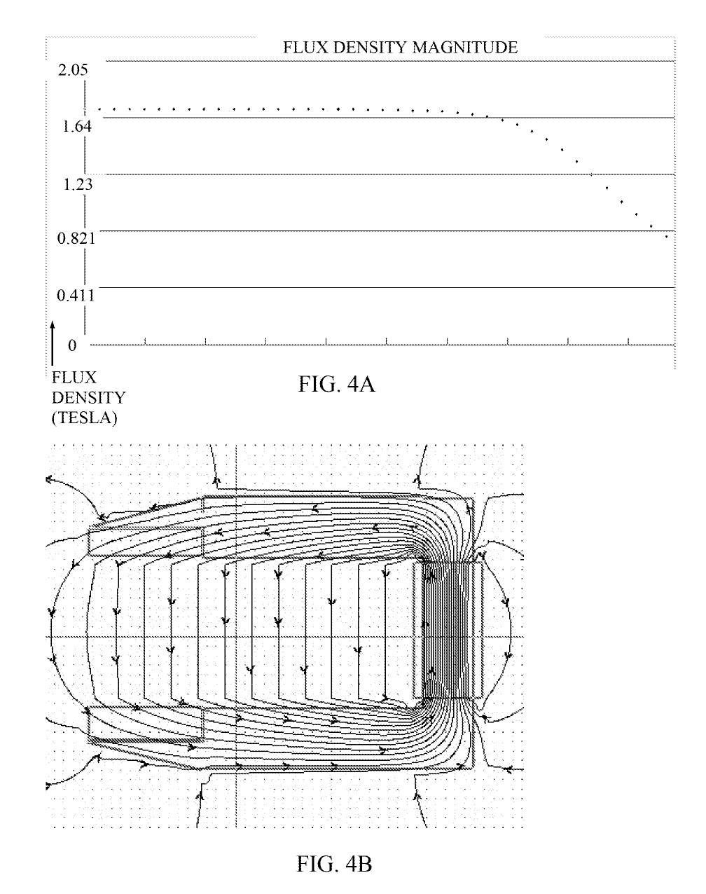

Thus, the system of FIG. 1 includes a patent table 29 where the patient is prone, stomach down. The breast hangs through the table into the magnet assembly 44. The pole pieces of the magnetic assembly 44 rest on the chest of the patient. FIG. 2 shows the location of the RF coils and magnetic working space below the table. FIG. 3 illustrates one embodiment for the construction of a magnet with a usable workspace. FIGS. 4A and 4B are plots of the raw static magnetic field.

In FIG. 3, apertures are provided for the breasts in the magnet assembly 44. RF field matching structure is provided to simulate an infinite volume to the RF coil assembly. The RF field matching structure may include a first inner layer 54 is provided adjacent the magnets and is made of an absorbent material, such as carbon-filled phenolic, and multilayers of material that have different dielectric constants so as to match the free air impedance to the impedance of the absorbent material. A second layer 56 is a vertical gradient field source, such as an arrangement of coils to generate a linear or pseudo-linear field in the vertical direction. A third layer 58 is a horizontal gradient field source, such as an arrangement of coils to generate a linear or pseudo-linear field in the horizontal direction.

Thus, the coil structure for use in producing MR images includes a gradient coil assembly for generating magnetic field gradients in two orthogonal planes, an RF coil assembly for transmitting or detecting a plurality of MR signals induced from the subject and a RF field termination assembly. In the present embodiment these coils have been integrated into a single set of quadrate coils.

The system may employ two receivers. However, it is to be appreciated that further embodiments may include multiple receivers, and image processing computations described below would be adapted by one skilled in the art for the selected receivers.

FIGS. 4A and 4B are plots of the flux density and flux lines, respectively, of the magnetic field strength in the gap, from base to pole piece tip. The magnetic field is from a single source and guided to the workspace over a magnetic path. The path supplies the weighting needed to provide the uniformity on the main magnetic field. In another embodiment, small gradient coils are added to allow shim and rotation to the magnetic fields.

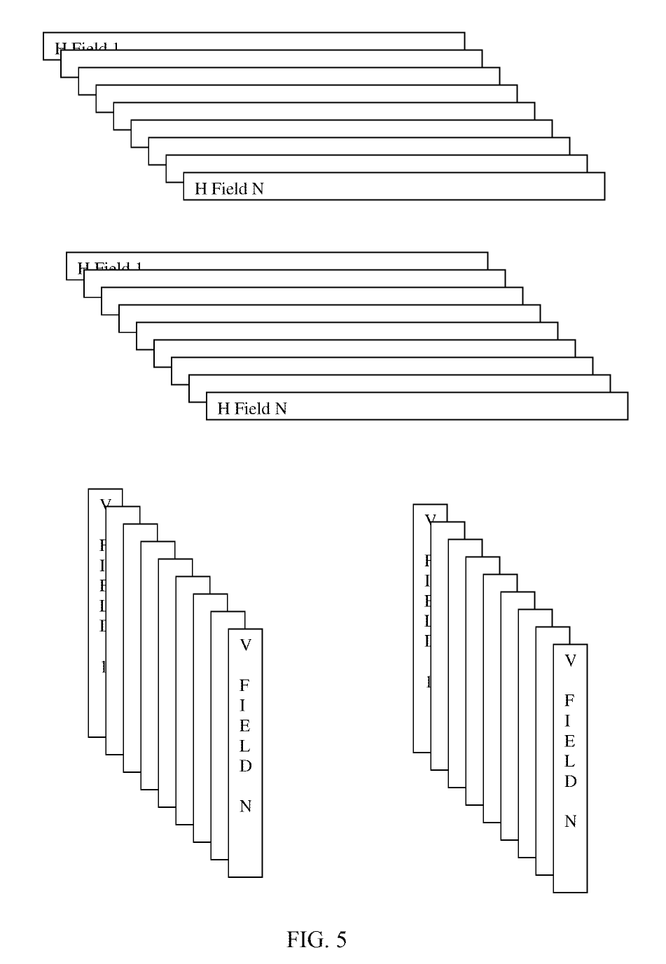

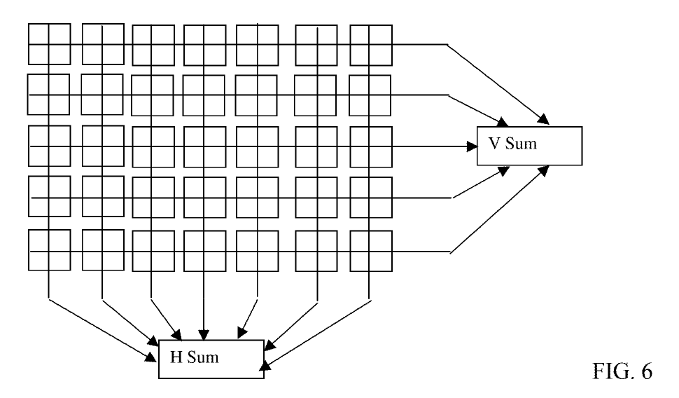

FIG. 5 is a representation of an orthogonal set of linear field coils for the generation of the necessary gradient fields. Small, low inductance, coils are utilized to allow for fast switching and field generation. In another embodiment, as in FIG. 6, a large number of small polar elements are located in programmable feed array. This configuration allows almost total flexibility of composite fields and shim correction in a single array. It is to be noted that a magnetic array exists on opposing sides of the working volume, in front of a magnetic medium and that the resulting field is linear.

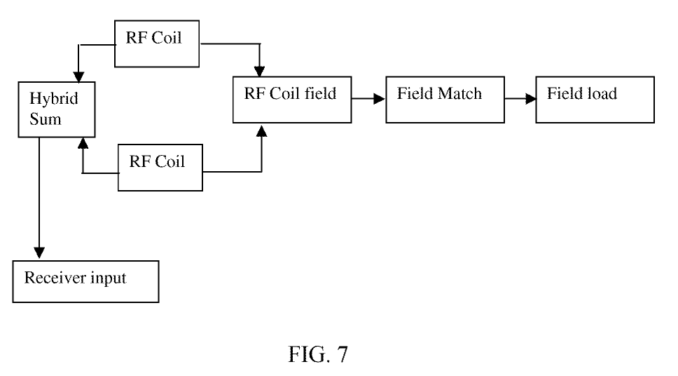

FIG. 7 is a representation of the RF field generation system (for each pole of the magnet). It is to be noted that the T/R switch is not shown since reciprocity is assumed. The NMR signal source is transmitted into the working volume and is received by the RF coils. At the same time, the signal is propagated through the entire volume, reflecting off each path discontinuity and creating image artifacts. To avoid this problem, a non-magnetic field termination system is placed in front of the metal surfaces. This termination system consists of a field transformation using graded dialectic materials and terminating loss material. The same system terminates the transmitted pulse, preventing extraneous NMR excitations.

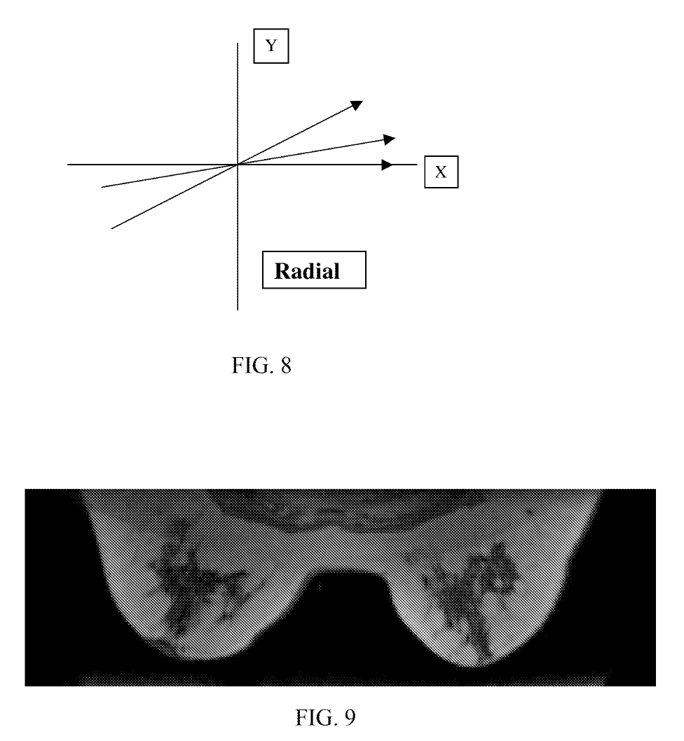

FIG. 8 is a representation of the so-called K-space. It should also be noted that oblique scanning is possible by performing simple matrix rotations to the gradient subsystem and to the receiver and transmitters frequencies in a manner well known to those skilled in the art. In this embodiment K-space is filled on radii. Since a single pixel at the center will input data from more than a single radial vector, the pixel input data is averaged. The average process also serves to reduce the noise on the intensity in transform space and no appreciable loss in edge quality.

It is appreciated that the method may be extended to multiple dimensions and/or simultaneous processing of multiple slices.

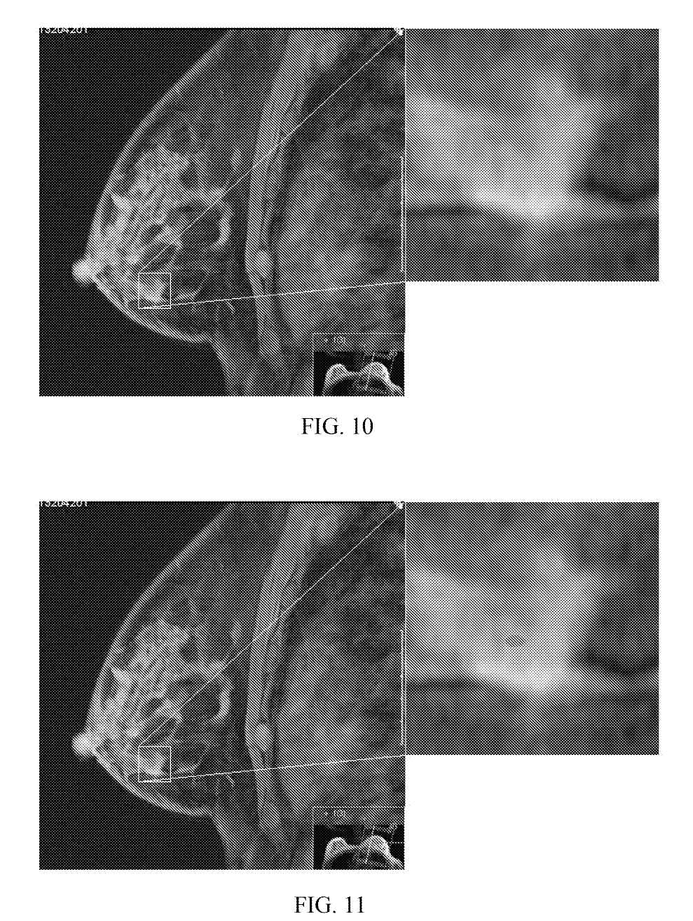

In this illustrative image, shown in FIG. 9, the transverse image represents both breasts in a view similar to a standard mammography image. In another embodiment only a single breast may be displayed. In this illustrative image, shown in FIG. 10, the sagittal image represents a single breast in a view orthogonal to a standard mammography image. The transverse image has been inserted digitally. The open magnet structure has made a MRI guided biopsy possible and this is shown in the image of FIG. 11.

A further alternate embodiment of the invention employs a magnet that is mounted on a stand instead of under a table. The stand provides the needed RF shielding. Such an embodiment is usable for treatment of extremities, such as arms, legs and joints, such as the knee, elbow and ankle.

Embodiments of the present invention provide for a MRI system in a confined volume using any MR pulse sequence. Embodiments of the methods of the present invention allow the user to select the desired direction of the phase and frequency encoding directions in order to advantageously place phase-encoding artifacts in desired directions. The user is also able to select interleaved acquisition of multiple slices, if necessary.

The scope of the present invention includes both combinations and subcombinations of the features described hereinabove as well as modifications and variations thereof which would occur to a person of skill in the art upon reading the foregoing description and which are not in the prior art.

What is claimed is:

1. Apparatus for producing magnetic resonance (MR) images of a subject over a limited volume, comprising:
a patient table; and
a shielded unit housed under said patient table, said shielded unit comprising an RF pulse generator module, a magnet assembly for producing a static magnetic field in magnetic structure comprising a shaped magnetic “C” core, a gradient field generating assembly for generating magnetic field gradients in two orthogonal planes for producing MR images, a radiofrequency (RF) coil assembly for transmitting a broad band radiofrequency pulse and a RF field matching structure to simulate an infinite volume to said RF coil assembly, wherein said RF coil assembly detects a plurality of MR signals induced from a subject in response to RF pulses from said RF pulse generator.

2. Apparatus according to claim 1, wherein said matching structure reduces the effect of reflections from said magnetic structure.

3. Apparatus according to claim 1, wherein said matching structure comprises a first layer adjacent the magnet structure and made of an absorbent material and multilayers of material that have different dielectric constants so as to match free air impedance to impedance of the absorbent material, a second layer which is a vertical gradient field source, and a third layer which is a horizontal gradient field source.

4. Apparatus according to claim 1, further comprising a plurality of digital receivers for receiving the plurality of MR signals.

5. Apparatus according to claim 4, wherein said digital receivers are adjusted in phase and frequency to produce parallel spectral levels as inputs to K-space.

6. Apparatus according to claim 5, further comprising a digital processor that transforms the K-space date to an image.

7. Apparatus for producing magnetic resonance (MR) images of a subject over a limited volume, comprising:
an operator console comprising a computer interface and a control system;
a patient table; and
a shielded unit housed under said patient table, said shielded unit comprising an RF pulse generator module, a magnet assembly for producing a static magnetic field in magnetic structure comprising a shaped magnetic “C” core, a gradient field generating assembly for generating magnetic field gradients in two orthogonal planes for producing MR images, a radiofrequency (RF) coil assembly for transmitting a broad band radiofrequency pulse and a RF field matching structure to simulate an infinite volume to said RF coil assembly, wherein said control system receives commands from an operator, the commands including a scan sequence that is to be performed on a subject, and wherein said control system controls operating parameters of said RF pulse generator module to carry out the scan sequence, and
wherein said RF coil assembly detects a plurality of MR signals induced from the subject in response to RF pulses from said RF pulse generator;
wherein said matching structure reduces the effect of reflections from said magnetic structure, and wherein said matching structure comprises a first layer adjacent the magnet structure and made of an absorbent material and multilayers of material that have different dielectric constants so as to match free air impedance to impedance of the absorbent material, a second layer which is a vertical gradient field source, and a third layer which is a horizontal gradient field source.

8. Apparatus according to claim 7, further comprising a plurality of digital receivers for receiving the plurality of MR signals.

9. Apparatus according to claim 8, wherein said digital receivers are adjusted in phase and frequency to produce parallel spectral levels as inputs to K-space.

10. Apparatus according to claim 9, further comprising a digital processor that transforms the K-space date to an image.
